(19) United States
(12) Patent Application Publication
(10) Pub. No.: US 2005/0267508 A1
(43) Pub. Date: Dec. 1, 2005

(54) Balloon catheter

(75) Inventors: Tomihisa Kato, Aichi-ken (JP); Fuminobu Yoshimachi, Aomori-ken (JP)

Correspondence Address: MORGAN LEWIS & BOCKIUS LLP, 1111 PENNSYLVANIA AVENUE NW, WASHINGTON, DC, 20004, US

(73) Assignee: Asahi Intec Co., Ltd.

(21) Appl. No.: 11/137,324

(22) Filed: May 26, 2005

(30) Foreign Application Priority Data
May 28, 2004 (JP) 2004-158549

(51) Int. Cl. A61M 29/00

(52) U.S. Cl. 606/192

(57) ABSTRACT

In a balloon catheter 1 in which a dilatable balloon portion 3 is secured to a distal end of a flexible and elongated shaft portion 2, at least two radiopaque ribbon markers M1, M2 are mutually spaced and provided on an outer surface of the elongated shaft portion 2 within the balloon portion 3. An inner interval L1 and an outer interval L2 between the ribbon markers M1, M2 are measurement rules determined by two different length units. The balloon catheter 1 contributes to treating a strictured area of the cardiovascular system with the use of the ribbon markers M1, M2, thus enabling manufacturers to put a drug-eluting stent into practical use which would be supposed to prevail widely in the medical field.

### BACKGROUND OF THE INVENTION

1. Field of the Invention

The invention relates to a balloon catheter used for medical treatment especially upon dilating a strictured area of the cardiovascular system.

2. Description of Related Art

A balloon catheter has a very thin and flexible hollow shaft portion, to a distal end of which a balloon portion is secured. The balloon catheter is inserted into a blood vessel to set the balloon portion in a strictured area of the cardiovascular system. Then, the balloon portion is manipulatively inflated to dilate the strictured area so as to retain an expandable stent within the dilated area upon treating the strictured area.

A variety of stents of different dimensions have been introduced, and scheduled to be delivered in the designs as shown in Table 1. It is possible to appropriately select a desired one from a group of stents in Table 1 in accordance with the specific feature of the cardiovascular disease.

TABLE 1

| Name of product | Maker's Name | Country | Length of stent |
|---|---|---|---|
| PALMAZ-SCHATZ ™ | Jonson & Jonson | US | 8.9 mm(0.35 inches), 15.0 mm(0.59 inches) |
| Stent | Medical Incorp. | | 13.8 mm(0.543 inches) |
| NIR ™ stent | Boston Scientific | US | 9 mm(0.354 inches), 16 mm(0.63 inches) |
| | | | 25 mm(0.984 inches), 32 mm(1.26 inches) |
| Terumo Stent | Terumo Incorp. | Japan | 20 mm, 30 mm, 40 mm |
| TENAX Stent | Biotronik Gmbh | Germany | 10 mm, 15 mm, 20 mm |
| | | | 25 mm, 30 mm, 35 mm |
| CYPHER Stent | Jonson & Jonson | US | 28 mm(1.10 inches) |
| Drug-Eluting Stent | Medical Incorp. | | 33 mm(1.30 inches) |

Laid-open Japanese Patent Application No. 2001-37882 (referred to as “a first document” hereinafter) and Domestically Open Japanese Patent Application No. 7-500749 (referred to as “a second document” hereinafter) disclose a balloon catheter and a guide wire for catheter in which the radioactive emission enables a manipulator to visually recognize how the catheter advances through the blood vessel.

The first document introduces the graduated lines to indicate an inserted length to recognize how far the balloon portion advances through the blood vessel, and the radiopaque markers are provided on the distal end of the shaft portion outside the balloon portion.

The second document introduces the radiopaque markers placed on the distal end of the catheter with the predetermined intervals as measurements.

In the meanwhile, since the stents in Table 1 have a variety of dimensional lengths and two different length units measured by inch and millimeter, it would be time-consuming and more troublesome to appropriately select the stent suitable for the cardiovascular disease. This would cause the manipulator to select the stent unsuitable for the individual diseases and wrongly retain the stent at the strictured area of the blood vessel.

The first document uses the markers to visually recognize only the distal end of the balloon catheter, and the second document employs the measurement markers with the measurement unit only confined to either inch or millimeter. None of the documents, accordingly, has no function supportable to appropriately select the desired one from the stents complicatedly classified as shown in Table 1.

Therefore, it is an object of the invention to overcome the above drawbacks, and provide a high quality balloon catheter which is capable of improving a treatment against the disease, and enabling manufacturers to put a drug-eluting stent into practical use which would be supposed to prevail in the medical field.

### SUMMARY OF THE INVENTION

According to the invention, there is provided a balloon catheter in which a distal end portion of a flexible and elongated shaft portion has a dilatable balloon portion. At least two radiopaque ribbon markers are mutually spaced and provided on an outer surface of the elongated shaft portion within the balloon portion.

When setting an inner interval as a distance between an inner edge side of one ribbon marker and an inner edge side of other ribbon marker, and setting an outer interval as a distance between an outer edge side of one ribbon marker and an outer edge side of other ribbon marker, the inner interval and the outer interval are measurement rules determined by two different length units.

With the balloon catheter according to the invention, the two intervals of the ribbon markers serve as the measurement rules by two different length units (inch and millimeter). This enables a manipulator to measure a diseased area by the two units, and making it easy to select a suitable one among stents of various sizes measured by two different length units so as to resultantly improve the treatment against the diseased area.

According to other aspect of the invention, a third ribbon marker is provided on the elongated shaft portion outside the balloon portion in an aim to significantly improve the treatment against the diseased area.

According to other aspect of the invention, the third ribbon marker is formed by a helical coil spring.

According to other aspect of the invention, an effective length of the balloon portion is less than 20 mm, and the inner interval or the outer interval between the one ribbon marker inside the balloon portion and other ribbon marker outside the balloon portion is more than 25 mm.

The balloon catheter is used as a pre-dilatation balloon catheter in the following manner.

(1) Upon retaining the stent, the pre-dilatation catheter together with a guide wire and a guiding catheter are used to introduce the balloon catheter into the diseased area with the assist of the guide wire.

(2) Before setting the stent, the balloon catheter is selected so that its dilatation diameter generally comes equal to a corresponding vascular diameter. The corresponding vascular diameter is calculated by a formula of {(proximal vascular diameter+distal vascular diameter)/2} in the diseased area. Then, the balloon catheter is dilatively pushed into the strictured area.

In this instance, a lengthwise relationship between the stent and the diseased area is visually recognized with the use of the ribbon markers, thus making it possible to select the most appropriate stent for the diseased area. After confirming the most appropriate stent, the balloon catheter is withdrawn.

(3) With the use of a stent-retaining balloon catheter, the balloon is dilatively pressurized to retain it in the diseased area. Thereafter, the balloon portion is shrinkably depressurized to withdraw the balloon catheter together with the guide wire and the guiding catheter. During these processes, it is possible to structurally recognize the diseased area with the use of the injected contrast medium and the intravenous ultrasonics (IVUS).

(4) Before retaining the stent, the radiopaque-marked balloon catheter is used to previously dilate the strictured area so as to make a smooth passage for the stent-retaining balloon catheter.

It is to be noted that the pre-dilatation catheter has an effective length of the balloon portion predetermined to be relatively small (e.g., 15-20 mm in length) to make its smooth passage into the strictured area, and making its ready dilatation from the distal end of the vascular portion when the diseased area is somewhat lengthened. The ready dilatation categorically includes a dilative ease felt when gradually dilating the blood vessel from the thin vascular portion, and including a manipulative ease felt when navigating the catheter from the sinuous path to the diseased area.

With the pre-dilatation catheter having ribbon markers marked by the two different measurement units, it is possible to readily recognize a suitable length of a stent and a lengthwise measurement of the diseased area depending on the condition of the diseased area when the length of the stent is marked by different measurement units.

(5) Then, the stent-retaining catheter is inserted to dilatably place the stent in the diseased area which was already dilated by the pre-dilatation catheter. After dilatively treat the diseased area, the stent-retaining catheter is withdrawn from the diseased area.

With the balloon catheter thus utilized as the pre-dilatation catheter, it is possible to dimensionally measure the intervals between paired ribbon markers by two different length units in terms of inch and millimeter. The radioactive emission enables the manipulator to implement the lengthwise measurements by either inch or millimeter. This makes it possible for the manipulator to recognize the most suitable stent precisely for the individual diseased areas.

With the measurements thus implemented, it is possible to readily select an appropriate one among the stents of various length sizes (see Table 1), and thus making it ready to select the desired stent with precise and quick procedures.

With the paired ribbon markers provided on the balloon catheter, it is possible to use four types (measuring rules) of combination regarding an inner edge side and an outer edge side of the paired ribbon markers, and thereby significantly improving a length measuring capability against the diseased area.

This facilitates to provide a quick and efficient treatment against the diseased area, and producing the most suitable selection among the stents of various length sizes.

### BRIEF DESCRIPTION OF THE DRAWINGS

Preferred forms of the present invention are illustrated in the accompanying drawings in which:

FIG. 1 is a longitudinal cross sectional view of a balloon catheter according to a first embodiment of the invention, but partly remaining as a side elevational view;

FIG. 2 is an explanatory view of the balloon catheter inserted into a diseased area;

FIG. 3 is a longitudinal cross sectional view of a balloon catheter according to a second embodiment of the invention;

FIG. 4 is an explanatory view of the balloon catheter inserted into a diseased area;

FIG. 5 is a longitudinal cross sectional view of a balloon catheter according to a third embodiment of the invention, but partly broken;

FIG. 6 is a longitudinal cross sectional view of a balloon catheter according to a fourth embodiment of the invention, but partly broken; and

FIG. 7 is a longitudinal cross sectional view of a balloon catheter according to a fifth embodiment of the invention, but partly broken.

### DETAILED DESCRIPTION OF THE PREFERRED EMBODIMENTS

In the following description of the depicted embodiments, the same reference numerals are used for features of the same type.

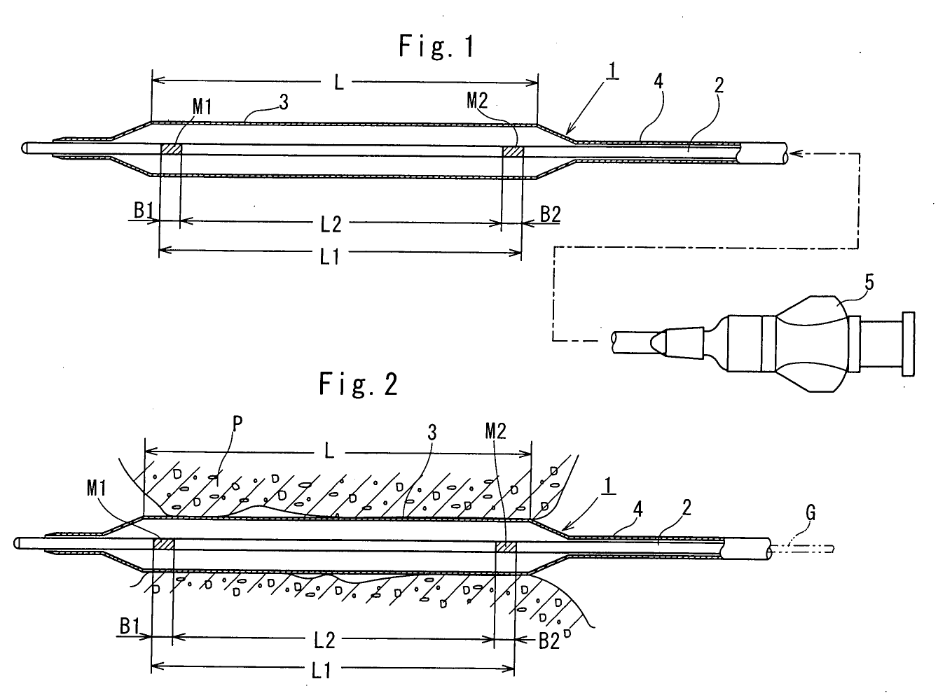

Referring to FIGS. 1 and 2, a balloon catheter 1 is provided according to a first embodiment of the invention. The balloon catheter 1 has a flexible and elongated shaft portion 2 around which a balloon portion 3 is provided. Into the shaft portion 2, a guide wire G is adapted to be inserted as shown at phantom lines in FIG. 2

An outer surface of the elongated shaft portion 2 has two radiopaque ribbon markers M1, M2 provided inside the balloon portion 3 in a mutually spaced relationship.

The ribbon marker M1 located at the distal side of the shaft portion 2 has a breadth B1 (B1=1.0 mm), and the ribbon marker M2 located at the proximal side of the shaft portion 2 has a breadth B2 (B2=1.2 mm).

An outer interval L1 is determined to be 20 mm between an outer edge side of the ribbon marker M1 and an outer edge side of the ribbon marker M2. An inner interval L2 is determined to be 0.7 inches (17.8 mm) between an inner edge side of the ribbon marker M1 and an inner edge side of the ribbon marker M2. An effective length L of the balloon portion 3 is approximately 20 mm determined for a smooth passage into a diseased area P. An outer shaft tube 4 extends axially from a proximal end of the balloon portion 3, and is provided around an outer surface of the shaft portion 2 to produce a drug-flowing clearance between the outer shaft tube 4 and the shaft portion 2. A manipulation portion 5 is provided at a proximal portion of the balloon catheter 1.

In this instance, the outer interval L1 serves as a measuring rule to measure the length by millimeter, and the inner interval L2 serves as a measuring rule to measure the length by inch.

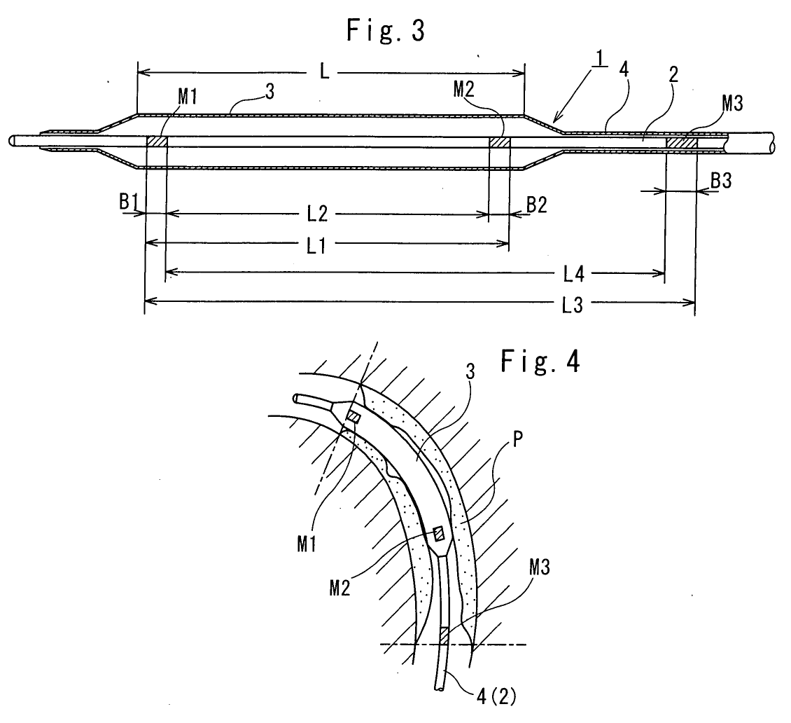

FIGS. 3 and 4 show a second embodiment of the invention in which a third ribbon marker M3 is provided on a proximal end of the shaft portion 2 outside the balloon portion 3 in addition to the ribbon markers M1, M2. Dimensions of these ribbon markers M1, M2, M3 are shown in Tables 2 and 3.

TABLE 2
For Markers M1, M2, M3, Inner Intervals L2, L4 Measured
by inch and Outer Intervals L1, L3 measured by mm
for Markers M1, M2, M3

| | breadth of | mutual interval | |
|---|---|---|---|
| Nos. | marker | of M1, M2 | mutual interval of M1, M3 |
| 1 | M1 = 1.0 mm | inner interval | inner interval L4 = 0.9 inches |
| | M2 = 1.2 mm | L2 = 0.7 inches | outer interval L3 = 25 mm |
| | M3 = 1.2 mm | outer interval | |
| | | L1 = 20 mm | |
| 2 | M1 = 1.0 mm | inner interval | inner interval L4 = 1.10 inches |
| | M2 = 1.2 mm | L2 = 0.7 inches | outer interval L3 = 30 mm |
| | M3 = 1.0 mm | outer interval | |
| | | L1 = 20 mm | |
| 3 | M1 = 1.0 mm | inner interval | inner interval L4 = 1.30 inches |
| | M2 = 1.2 mm | L2 = 0.7 inches | outer interval L3 = 35 mm |
| | M3 = 1.0 mm | outer interval | |
| | | L1 = 20 mm | |

TABLE 3
For Markers M1, M2, M3, Inner Intervals L2, L4 Measured
by inch and Outer Intervals L1, L3 measured by mm
for Markers M1, M2, M3

| | | mutual interval | |
|---|---|---|---|
| Nos. | breadth of marker | of M1, M2 | mutual interval of M1, M3 |
| 1 | M1 = 1.0 mm | inner interval | inner interval L4 = 25 mm |
| | M2 = 1.8 mm | L2 = 20 mm | outer interval L3 = 1.1 inches |
| | M3 = 1.9 mm | outer interval | |
| | | L1 = 0.9 inches | |
| 2 | M1 = 1.0 mm | inner interval | inner interval L4 = 30 mm |
| | M2 = 1.8 mm | L2 = 20 mm | outer interval L3 = 1.3 inches |
| | M3 = 2.0 mm | outer interval | |
| | | L1 = 0.9 inches | |

Upon using a drug-eluting stent (33 mm or 1.30 inches in length) which is useful to treat a strictured area and supposed to prevail, the balloon catheter 1 can be selected which has the ribbon markers as shown No. 3 in Table 2 or ones as shown No. 2 in Table 3.

In this instance, regardless of whether the drug-eluting stent is in terms of inch or millimeter, the outer and inner intervals are almost equal to a length (33 mm) of the drug-eluting stent, thus helping to precisely measure the diseased area P so as to help prevail the drug-eluting stent more widely.

In Tables 2 and 3, the outer intervals L3 of the ribbon markers M1, M2 may be determined to be more than 25 mm as a modification form. In the modification form, the balloon catheter 1 acts as a pre-dilatation balloon catheter to retain a lengthened stent.

The following are supplementary analyses regarding the balloon catheter provided for retaining a lengthened stent.

With the use of the balloon catheter according to the above modification form, it is possible to appropriately retain a lengthened stent of more than 25 mm (especially, drug-eluting stent) because the balloon catheter enables the manipulator to visually recognize the length of the lengthened stent irrespective of whether the stent is measured by inch or millimeter.

Upon retaining the stent, it poses the problems as follows:

It is desirable to fully cover the diseased area P with a single one stent. This is because the vascular diameter becomes thinner at the distal side as the diseased area P gets longer in size, the diseased area P may be dissociated as a result that the diseased area P is excessively dilated when equally dilated to the full length.

In order to avoid the excessive dilatation, a plurality of stents can be used. The stents, however, induces to occlude the diseased area again at the connection between the neighboring stents. Because this is all the more true for a semi-chronical disease (20 mm or more in length), it requires more sophisticated technique to retain the plurality of stents.

When the diseased area P is sinuous, the stent is subjected at both ends to pressure fluctuations due to the blood stream and its pulsation so that the diseased area P is liable to occlude again.

As for a fully occluded area, it is supposed that diseased areas likely appear in the neighborhood of the fully occluded area. This makes the diseased area longer in size, and often accompanies a large amount of plaques.

Since the intravenous ultrasonics (IVUS) brings information about the cross sectional shape of the fully occluded area at both ends and locations where the plaques and the thrombi appear, a stent is carefully selected based on the information derived from the intravenous ultrasonics (IVUS). The likelihood is reportedly as high as 30% that the diseased area P is occluded again in six months after the operation.

With the use of the balloon catheter 1 in which the three ribbon markers M1, M2, M3 are provided as shown in FIG. 3, it is possible to confirm whether or not the stent is appropriate to use for the balloon catheter 1 which has the outer and inner intervals L1, L2, L3, L4 determined between the ribbon markers M1, M2, M3. The balloon catheter 1 enables the manipulator to obtain a real length of the diseased area P in which the stent is to be retained, thus making it possible to quickly select the most suitable stent precisely in accordance with the length of the diseased area P. With the stent selected to be suitable for the diseased area P, it is possible to quickly retain the stent in the diseased area P with high precision so as to significantly improve the treatment against the diseased area P.

In the meanwhile, the drug-eluting stent has a first hydrophilic layer on a substrate (braided wire of stainless steel) and an outer hydrophobic layer which has a hardness greater than the first hydrophilic layer has. The first hydrophilic layer includes a medicinal substance, and the dilatation causes cracks to appear on the outer hydrophobic layer so as to gradually release a medicinal substance from the first hydrophilic layer.

When it takes longer to retain the stent on the diseased area P upon using the drug-eluting stent because the length of the stent is inappropriate for the diseased area P, the stent causes to rub the first hydrophilic layer against the calcified portion of the diseased area P so as to induce cracks on the first hydrophilic layer. The cracks cause to release the liquid medicine from the first hydrophilic layer to the blood steam, thus producing a systemic side effect to fail the medication for the diseased area P (inappropriate local medication or shortage of medication). This is all the more true especially upon using a lengthened stent.

With the use of the topography based on the intravenous ultrasonics (IVUS) as shown in FIG. 4, the manipulator selects an appropriate stent and the location in which the stent is to be retained. This is done by recognizing positions and directions in which the thrombus formation and the calcified plaque (soft plaque) are placed while taking into consideration a likelihood of the superficial calcification and structural ends of the diseased area P.

The location of the stent, however, is not always appropriate for the diseased area P under the presence of a thickness of the intravenous ultrasonics (IVUS), the vascular diameter, a thickness of the stent and a passage path of the diseased area P. Taking it into account that the stent is dilatively manipulated in one direction only once, it is necessary to use the pre-dilatation balloon catheter to confirm whether or not the length of the selected stent is appropriate.

Upon inserting the balloon catheter 1 into the diseased area P as the pre-dilatation balloon catheter, it dilates the diseased area P so that the pre-dilatation balloon catheter is appropriately set in the diseased area P. Then, the diseased area P teaches a required length and the location of the stent with the use of the ribbon markers M1, M2, M3. This makes it easy to select the stent depending on its length while making it ready to precisely recognized the location in which the stent is to be retained.

With the three ribbon markers M1, M2, M3 provided on the balloon catheter 1, it is possible to use a maximum interval between the ribbon markers M1 and M3 so as to extremely precisely measure a maximum diseased area in a single one span. This makes it possible to precisely measure the individual diseased areas while enabling the manipulator to select an appropriate one among the stents of two different length units (inch and millimeter), thus making it ready to correctly place the stent in the diseased area.

Under the environment that the likelihood is reportedly high that the diseased area P is occluded again, it is certain that the introduction of the drug-eluting stent makes it possible to drastically reduce the likelihood that the diseased area P is occluded again.

Upon retaining the drug-eluting stent with the use of the balloon catheter 1, the following advantages are obtained.

The balloon catheter 1 makes it possible to readily select an appropriate one having the length suitable for the diseased area among the drug-eluting stent of two different length units (inch and millimeter). If the selected stent is found inappropriate, the balloon catheter 1 makes it ready to replace the inappropriate stent with new one which has a length and a length unit other than the selected stent has.

Upon using the drug-eluting stent, the stent is generally placed so that it fully covers the diseased area to extend beyond both the ends of the diseased area. This situation has a tendency to make the stent lengthened so long as 25 mm. The balloon catheter 1 makes it possible to precisely retain the stent (25 mm or longer) with good drug-eluting capability maintained. This makes it ready to retain the drug-eluting stent and stabilize its good performance so as to prevail it more widely in the medical field.

It is to be noted that the ribbon markers may be formed by radiopaque cylinders. The cylinders are fit into the corresponding locations of the elongated shaft portion 2. Alternatively, the ribbon markers may be provided by means of sputtering or deposition to have a predetermined breadth and thickness (20-100 μ) with an effective radiopaqueness into consideration.

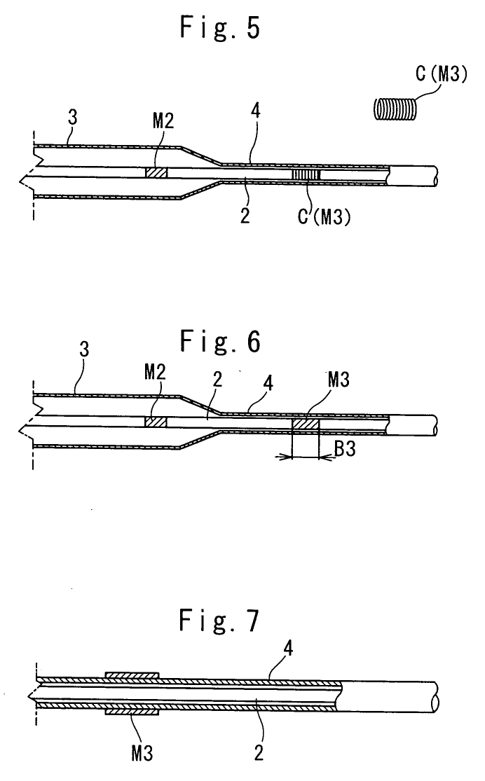

FIG. 5 shows a third embodiment of the invention in which the third ribbon marker M3 is made of a helical coil spring C. As shown in FIG. 5, the helical coil spring C is formed by winding a very thin wire of gold, platinum or tungsten (0.06 mm in diameter), and placed within the corresponding location of the elongated shaft portion.

In this instance, the ribbon marker M3 is superior in flexibility so that the ribbon marker M3 can compensate the loss of the flexibility of the elongated shaft portion 2. The ribbon marker M3 is readily produced by cutting a long helical coil spring at appropriate length sections.

FIG. 6 shows a fourth embodiment of the invention in which the ribbon marker M3 has a breadth B3 (B3=5.0 mm).

Upon selecting the stent (28 mm and 33 mm in length), a difference of which is 5.0 mm as understood by Table 1, it is possible to quickly obtain an appropriate one by using the intervals of the marker M3 against the markers M1, M2.

FIG. 7 shows a fifth embodiment of the invention in which the third ribbon marker M3 is provided on an outer surface of the outer shaft tube 4. In this instance, the breadth of the ribbon markers M1, M2, M3 can be altered as desired. Alternatively, it is to be noted that the ribbon markers M1, M2 can be divided into three sections inside the balloon portion 3.

1. A balloon catheter in which a distal end portion of a flexible and elongated shaft portion has a dilatable balloon portion,
said balloon catheter comprising:
at least two radiopaque ribbon markers mutually spaced and provided on an outer surface of said elongated shaft portion within said balloon portion;
an inner interval being between an inner edge side of one ribbon marker and an inner edge side of other ribbon marker;
an outer interval being between an outer edge side of one ribbon marker and an outer edge side of other ribbon marker; and
said inner interval and said outer interval being measurement rules determined by two different length units.

2. The balloon catheter according to claim 1, wherein one of said inner interval and said outer interval of said elongated shaft portion are measured by millimeter, and other marker measured by inch.

3. The balloon catheter according to claim 1 or 2, wherein at least one of said ribbon markers is provided on said elongated shaft portion outside said balloon portion, or provided on an outer shaft portion extending from said balloon portion rearwardly along said elongated shaft portion; and
said inner interval and said outer interval between said ribbon marker and other ribbon marker inside said balloon portion are measurement rules determined by said two different length units.

4. The balloon catheter according to claim 3, wherein said ribbon marker provided outside said balloon portion is formed by a helical coil spring.

5. The balloon catheter according to claim 3, wherein an effective length of said balloon portion is less than 20 mm, and said inner interval or said outer interval between said ribbon marker inside said balloon portion and other ribbon marker outside said balloon portion is more than 25 mm.

6. The balloon catheter according to claim 4, wherein an effective length of said balloon portion is less than 20 mm, and said inner interval or said outer interval between said ribbon marker inside said balloon portion and other ribbon marker outside said balloon portion is more than 25 mm.
